(19) United States
(12) Patent Application Publication
(10) Pub. No.: US 2016/0367169 A1
(43) Pub. Date: Dec. 22, 2016

(54) METHOD AND MEDICAL IMAGING APPARATUS FOR SELECTION OF AT LEAST ONE ITEM OF EXAMINATION INFORMATION FOR A MEDICAL IMAGING EXAMINATION

(71) Applicant: Siemens Healthcare GmbH, Erlangen (DE)

(72) Inventors: Donald Hardie, Moehrendorf (DE); Anja Jaeger, Fuerth (DE); Stephan Nufer, Erlangen (DE); Thorsten Speckner, Erlangen (DE); Jens Thoene, Nuernberg (DE)

(73) Assignee: Siemens Healthcare GmbH, Erlangen (DE)

(21) Appl. No.: 15/185,580

(22) Filed: Jun. 17, 2016

(30) Foreign Application Priority Data
Jun. 17, 2015 (DE) 102015211148.2

(51) Int. Cl. A61B 5/11; A61B 6/03; A61B 6/00; A61B 5/00

(52) U.S. Cl. CPC A61B 5/1113; A61B 5/0077; A61B 6/037; A61B 5/1128; A61B 5/7475; A61B 5/743; A61B 6/54; A61B 2090/374

(57) ABSTRACT

In a method and apparatus for the selection of at least one item of examination information for a medical imaging examination before the medical imaging examination begins, position data of the patient are detected using a position detector. For this purpose, the patient is disposed for the medical imaging examination on a patient support of a medical imaging device. The detected position data are evaluated by a position data evaluation processor, with a patient image that shows a surface image of the patient being created on the basis of the detected position data. The position data evaluation processor determines multiple items of examination information on the basis of the surface image. The multiple items of examination information are presented at an output interface. A selection of at least one item of examination information for the impending medical imaging examination is then made from the multiple displayed items of examination information.

### BACKGROUND OF THE INVENTION

Field of the Invention

The present invention concerns a method for selecting at least one item of examination information for a medical imaging examination before the beginning of the medical imaging examination. Furthermore the present invention concerns a medical imaging device with a scanner, a position detector, by which position data of a user are detected, a user interface that includes a display unit and that is disposed within an examination room, and a position data evaluation processor, wherein the position data evaluation processor, together with the position detector and the user interface, is designed to carry out a method for selection of at least one item of examination information for a medical imaging examination before the medical imaging examination begins.

Description of the Prior Art

Before a medical imaging examination is started, the patient must be supported and/or positioned correctly on a patient support. During this preparation of the patient, under some circumstances additional units can be attached to the patient for the medical imaging examination, such as an ECG unit and/or local radio-frequency antenna units, and/or further additional units required for the impending medical imaging examination. Furthermore, as part of such a patient preparation, patient-specific and/or examination-specific examination information and/or parameters may to have to be selected by medical operating personnel. This patient preparation is especially time-consuming and, depending on the level of skill of operating personnel, can lead to errors.

### SUMMARY OF THE INVENTION

An object of the present invention is to support medical operating personnel by providing an especially simple patient preparation can be provided.

One aspect of the invention is a method for the selection of at least one item of examination information for a medical imaging examination before the medical imaging examination begins, which starts by detection of position data of the patient using a position detector. For this purpose, the patient is disposed for the medical imaging examination on a patient support of a medical imaging device. The detected position data are evaluated by a position data evaluation processor, with a patient image that shows a surface image of the patient being created on the basis of the detected position data. The position data evaluation processor determines multiple items of examination information on the basis of the surface image. The multiple items of examination information are presented at an output interface. A selection of at least one item of examination information for the impending medical imaging examination is then made from the multiple displayed items of examination information.

In this context, position data of the patient means data representing position information about a position of the patient, and/or from which a position of the patient can be computed. Preferably the position data represent position information of the entire patient. The position of the patient is preferably to be determined and/or able to be determined in relation to the patient support, usually a horizontal table of the patient support, on which the patient is already positioned and/or disposed during the detection of the position data. The patient support thus preferably has such a horizontal table on which a patient can be supported and/or positioned for the medical imaging examination. From the detected position data, a surface image of the patient is created, on the basis of which information about a location and/or anatomy and/or a contour of the patient and/or of individual regions of the body of the patient can be obtained.

An item of examination information as used herein means information that is relevant for the impending medical imaging examination. The determination of a number of items of examination information includes the presentation of a number of items of examination information, from which the user, especially the medical operating personnel, can select at least one item of examination information. The number of examination parameters is preferably determined independently and/or automatically by the position data evaluation processor. The number of items of examination information can be, for example, different regions of the patient's body, and the user can select a region relevant to the examination from the different regions of the body. As an alternative or in addition, the number of items of examination information can be, for example, a maximum area specification for an overview measurement, and the user can select an overview area relevant for the impending medical imaging examination. In addition, the number of items of examination information can include assistance and/or instructions for next workflow steps in the patient preparation.

The presentation of the number of items of examination information can be a graphical presentation and/or a display of the number of items of examination information on a display monitor and/or a presentation unit. The at least one item of examination information is preferably selected manually by the user, by the user making a corresponding entry here is detected by an input interface. The output interface and the input interface are preferably disposed within an examination room, in which the scanner of the medical imaging device is also disposed. The position data evaluation processor is preferably disposed within a supervision room. An examination room as used herein means a room in which the medical imaging device, especially the scanner of the medical imaging device, is disposed. Preferably the examination room is screened to the outside and to the inside in order to screen out disruptive influences that can both influence a medical imaging examination and can also be caused by the medical imaging device. For example the examination room, for an embodiment of the medical imaging device as magnetic resonance device, is screened to the outside with respect to magnetic fields and is screened against electromagnetic radiation, especially radio-frequency magnetic radiation, to the outside and to the inside. In addition, for an embodiment of the medical imaging device as a computed tomography device, the examination room can be screened to the outside with respect to electromagnetic radiation.

The inventive method enables a patient preparation for a medical imaging examination to be advantageously supported. In particular the medical operating personnel can be supported here to the extent that, by the selection of an item of examination information from the number of items of examination information, additional information can be displayed as a function of the selected examination information.

In a further embodiment of the invention, output interface is a touch-screen display. In this way information can be displayed and parameters and/or positioning information for medical operating personnel can be entered especially easily. The touch-screen display can be disposed directly on the medical imaging scanner and/or on the patient support, so that medical operating personnel do not have to leave the examination room, in which the scanner of the medical imaging device is situated, during the preparation of the patient.

A touch-screen display is understood as a touch-sensitive screen, via which, by touching sub-areas of the screen, especially sub-areas of a displayed image on the screen, parameters can be entered, in order to control a program execution sequence, for example.

In a further embodiment of the invention, the output interface includes a projector and the presentation of the number of items of examination information is a projection of the number of items of examination information on a projection surface. In this context, a projection surface is understood as a surface that is designed such that at least one display object will be projected on the surface. The projection surface can be, for example, a surface of the housing of a scanner of the medical imaging device. Especially advantageously, however, the projection surface is a surface of the patient, so that the display objects can be projected directly into a working area and/or within a field of view of the medical operating personnel. Furthermore, a projector is understood as a component that is designed for projection of at least one display object onto the projection surface.

In a further embodiment of the invention, the projection of the number of items of examination information onto the patient is implemented true-to-scale for at least one of the number of items of examination information. For example, for a segmentation of the surface image into individual regions of the body, these individual regions of the body can be displayed superimposed directly onto the patient by the projector. This achieves a simple and direct selection of a region of the body for the medical imaging examination. In addition, with a selection of an area of an overview measurement, a maximum area can be displayed directly on the patient true-to-scale, so that the area of the overview measurement can be defined directly on the patient. In addition, an area of the patient, which is to be disposed in an isocenter of the scanner for the impending medical imaging examination, is projected directly onto an examination area of the patient, so that for operating personnel a direct and simple check of a positioning of the area of the patient can be achieved in relation to the isocenter area. During the medical imaging examination, the isocenter area should coincide at least partly with the isocenter of the scanner of the medical imaging device.

In a further embodiment of the invention, the selection of at least one item of examination information detected by the position detector, is made by a gesture of a user on and/or at the projection surface. In this way an especially simple and time-saving selection of the at least one item of examination information can be made, since the user does not have to leave a current working position in order to do so, for example with a projection directly onto the patient. This makes it possible for medical operating personnel to make a direct selection, without having to interrupt a current activity to do so. Preferably the position data evaluation processor is designed so as to differentiate a selection gesture of the operator from a random gesture made in the projection area, so that a selection made by the operator can be reliably detected.

In a further embodiment of the invention, the number of items of examination information is presented as text information and/or as graphic symbols for a user. In this way, the output or presentation can be adapted to a user. In addition, the display of the number of items of examination information as graphic symbols advantageously makes it possible for the number of items of examination information to be presented in a manner that is comprehensible in any language.

In a further embodiment of the invention, dependent on the at least one selected item of examination information, information relevant for the medical imaging examination is displayed for a user. In this way, a pre-selection, which is made dependent on the at least one selected item of examination information, can already be displayed to the user, so that the further preparation steps, such as a selection of examination protocols and/or an entry of a patient position and/or attachment of additional units to the patient etc. can be done in an especially time-saving manner. In addition, the information relevant for the medical imaging examination can include additional instructions or assistance to a user as to how to proceed further.

In a further embodiment of the invention, the information relevant for the medical imaging examination includes assistance for preparation of the medical imaging examination. In this context assistance for preparation of the medical imaging examination is understood as information that includes assistance and/or an instruction and supports the medical operating personnel in a work sequence of the preparation of the medical imaging examination on the patient. This embodiment of the invention makes possible a simple and guided patient preparation, in which even unskilled operating personnel can be guided through the workflow. In addition advantageous support can also be provided even for medical imaging examinations that are not covered by routine examinations, so that errors in their preparation can be prevented.

In a further embodiment of the invention, the information relevant for the medical imaging examination includes at least one examination protocol. Preferably the information relevant for the medical imaging examination includes a selection of relevant examination protocols, wherein the selection of the relevant examination protocols can be adapted, for example, to a selected region of the body. This enables the selection of one or more examination protocols necessary for the preparation of the medical imaging examination to be made in an especially time-saving manner, since only a limited selection of examination protocols is now available in accordance with the invention. In addition, as a result of the limited selection of examination protocols, the probability of an incorrect selection of an examination protocol is reduced.

In another embodiment of the invention, the information relevant for the medical imaging examination includes at least one item of information of an additional unit that is to be positioned on the patient for the medical imaging examination. Such an additional unit can be, for example, an ECG unit, an injector unit, or a local radio-frequency coil unit. The information of the additional unit here can include a type of the additional unit and/or an item of positioning information of the additional unit and/or an item of coupling information of the additional unit, etc. This embodiment of the invention makes possible a simple and guided fitting of additional units on the patient, in which even untrained operating personnel can advantageously be guided through the workflow. In addition, the medical operating personnel can be advantageously alerted about possible sources of error during the attachment of the additional units, for example connection of additional units to the medical imaging device before the medical imaging examination.

In a further embodiment of the invention, the information relevant for the medical imaging examination includes an examination region of the patient. In this way the examination region can already be defined during the preparation phase by the user. A later definition of the examination region, which is done outside the examination room in a supervision room at an operator console, thus can be dispensed with. This makes practical support of the operator in the preparation possible as a result of the definition of the examination region. In this context, an examination region is understood as a region of the patient's body at which the medical imaging examination is to take place. Preferably the examination region is defined by the surface image of the patient. This examination region can include an anatomically-delimited area of the patient, such as the legs of the patient, the arms of the patient, or the head of the patient, or the torso of the patient, etc. The definition of the examination region can also include a defined area specification for an overview measurement.

In another embodiment of the invention, the at least one item of examination information delimits an examination region in at least one dimension. At least one dimension is understood as a length and/or a width and/or a height of the examination region. The examination region can be delimited by selection of a region of the patient's body, such as selection of the patient's torso. It is also conceivable for the delimitation of the examination region to include an area definition for an overview measurement, for example. This makes it possible for the examination to be commenced in the examination room immediately after the preparation phase and not, as previously, with a relevant area and/or relevant settings having to be defined in a supervision room.

Preferably the at least one item of examination information defines a delimitation of an examination region for an overview measurement. This makes possible an accelerated work sequence during a preparation phase for the medical imaging examination.

In a further embodiment of the invention, the surface image is segmented by the position data evaluation processor and the number of items of examination information includes segment areas of the segmented surface image. This makes possible an especially simple selection of the examination area for an operator, since the operator only has to select one segmented area via the input unit. This can be achieved when the selection of that at least one item of examination information includes a selection of a segment area of the segmented surface image.

In another embodiment of the invention, different segment areas of the segmented surface image are marked differently. Preferably the individual marking areas are marked automatically and/or independently by the position data evaluation processor. The different markings of the different segment areas can be, for example, different colors and/or different shadings, etc. In this way an especially simple overview over the surface image for a user, especially for medical operating personnel, can be achieved. This makes possible an especially time-saving selection of a segment area and thus of a region of the body during the preparation of the medical imaging examination.

In another embodiment of the invention, a definition of an isocenter area for the medical imaging examination is made on the basis of the surface image of the patient. The isocenter area defines an area of the patient that coincides at least partly with the isocenter of the scanner of the medical imaging device for the impending medical imaging examination. The definition of the isocenter area on the patient, when the latter is still disposed outside the detector unit, contributes to a feeling of wellbeing of the patient, since the patient then only has to be introduced into a patient receiving area for the duration of the medical imaging examination. The more relaxed the patient is for the impending medical imaging examination, the easier it is for medical imaging data with a high image quality to be obtained.

In this context, an isocenter is understood as a point and/or an area that is located within a patient receiving area and in which, within the medical imaging device, the most ideal conditions for the medical imaging examination exist during operation of the medical imaging device. Preferably the isocenter describes a point within an area that includes the isocenter. For example, the isocenter within a magnetic resonance device includes a point and/or an area at which the magnetic field of the magnetic resonance scanner is at its most homogeneous, and that is preferably disposed in a center of the basic field magnet and/or a gradient coil unit. The isocenter for a medical imaging device is preferably defined once during the installation of the medical imaging device.

The invention further encompasses a medical imaging device with a scanner, a position detector, with which position data and/or movement data of a user are detected, a user interface, which includes an output interface that is disposed within an examination room, and a position data evaluation processor, wherein the position data evaluation processor is designed, together with the position detector and the user interface, to carry out the inventive method for selection of at least one item of examination information for a medical imaging examination.

The inventive apparatus enables the patient preparation for a medical imaging examination advantageously to be supported. In particular the medical operating personnel can be supported to the extent that, through the selection of an item of examination information, additional information can be presented dependent on the selected examination information.

The medical imaging apparatus can be any medical imaging device appearing sensible to those skilled in the art. Preferably the medical imaging apparatus is a magnetic resonance apparatus and/or a computed tomography device and/or a PET (Positron Emission Tomography) device.

The advantages of the inventive medical imaging apparatus essentially correspond to the advantages of the inventive method for selection of at least an item of examination information for a medical imaging examination, as described in detail above. Features, advantages or alternate forms of embodiment mentioned above are applicable to the apparatus.

In an embodiment of the presentation unit includes a projector that is designed to project on a projection surface. In this way an especially user-friendly display can be provided for medical operating personnel, since the projection surface can be disposed in a field of view and/or in a working area of the user. Interrupting an operating step, especially in order to note information shown on a display or a monitor, is thus not necessary.

In a further embodiment of the apparatus of the invention, the presentation unit has a projection surface that includes a housing of the scanner or a patient surface. In this way information can be displayed within the examination room especially easily, so that the user, especially one of the medical operating personnel, does not have to leave the examination room to obtain information while preparing the patient for the medical imaging examination.

As an alternative or in addition, the presentation unit can include a touch-screen display. This makes possible an especially simple display of information and also makes possible an entry of parameters and/or positioning information for medical operating personnel.

In a further embodiment of the invention, the position detector is a 2D camera or a 3D camera for detection of two-dimensional position data or for detection of three-dimensional position data. In this way an especially simple and cost-effective position detector can be provided.

The invention further encompasses a non-transitory, computer-readable data storage medium encoded with program code, the storage medium being loaded directly into a memory of a programmable position data evaluation processor of a medical imaging device. The program code causes the processor to carry out the method for selection of at least one item of examination information for a medical imaging examination in accordance with the invention, when the program code is executed in the position data evaluation processor. The program code may possibly need peripheral means, e.g. libraries and auxiliary functions, in order to realize the corresponding embodiments of the method. The program code can be software with a source code that still has to be compiled and linked, or only has to be interpreted, or can be an executable software code that still has to be loaded into the corresponding processing unit for execution.

### BRIEF DESCRIPTION OF THE DRAWINGS

FIG. 1 shows a first inventive medical imaging device in a schematic illustration.

FIG. 2 shows a second inventive medical imaging device in a schematic illustration.

FIG. 3 is a flowchart of an inventive method for selection of at least one item of examination information for a medical imaging examination.

FIG. 4 shows a first exemplary embodiment of a display of examination parameters.

FIG. 5 shows a second exemplary embodiment of a display of examination parameters.

### DESCRIPTION OF THE PREFERRED EMBODIMENTS

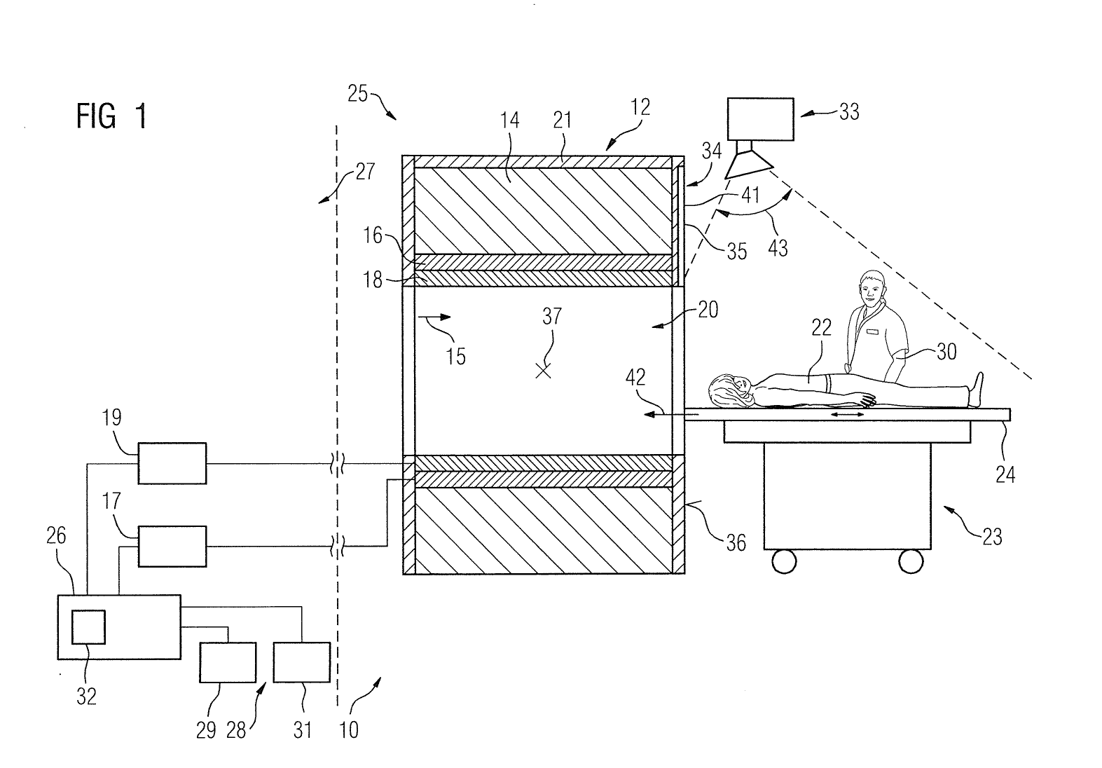

FIG. 1 is a schematic illustration of a medical imaging apparatus 10. In the present exemplary embodiment the medical imaging apparatus 10 is formed by a magnetic resonance apparatus. The embodiment of the medical imaging apparatus 10 is, however, not restricted to a magnetic resonance apparatus, but the medical imaging apparatus 10 can be formed by any medical imaging apparatus appearing reasonable to those skilled in the art, such as a computed tomography device, a positron emission tomography device, etc.

The medical imaging apparatus 10 includes a medical image data acquisition scanner 12, which in the present exemplary embodiment is formed by a magnet unit of the magnetic resonance apparatus. The magnet scanner 12 includes a superconducting basic field magnet 14 for creating a strong and constant basic magnetic field 15. Furthermore the scanner 12 has a gradient coil arrangement 16 for creating magnetic field gradients that are used for spatial encoding during imaging. The gradient coil arrangement 16 is controlled by a gradient control processor 17 of the magnetic resonance scanner 12. The scanner 12 also includes a radio-frequency (RF) antenna 18 for exciting nuclear spins in the patient 22 so as to deviate from the polarization that arises in the basic magnetic field 15 created by the basic field magnet 14. The radio-frequency antenna 18 is controlled by a radio-frequency antenna processor 19 of the magnetic resonance apparatus so as to emit radio-frequency magnetic resonance sequences into a patient receiving area 20 of the scanner 12.

The scanner 12 is surrounded by a housing 21.

The medical imaging apparatus 10, in the present exemplary embodiment the magnetic apparatus, also has a patient receiving area 20 for receiving the patient 22. The patient receiving area 20 has a cylindrical shape in the exemplary embodiment and is surrounded in a circumferential direction by the scanner 12. Basically, however, an embodiment of the patient receiving area 20 differing from this embodiment is conceivable. The isocenter 37 of the magnetic resonance scanner 12 is disposed within the patient receiving area 20.

The patient 22 can be moved into the patient receiving area 20 by a patient support 23 of the medical imaging apparatus 10. The patient support 23 has a patient table 24 mounted to allow movement within the patient receiving area 20.

The scanner 12 is disposed, together with the housing 21 and the patient support 23, within an examination room 25. The examination room 25 is screened with respect to electromagnetic radiation and/or with respect of propagation of magnetic fields. For controlling the magnetic resonance scanner 12, such as for controlling the basic field magnet 14, the gradient control processor 17 and the radio-frequency antenna control processor 19, the medical imaging device 10 has a system control computer 26. The system control computer 26 is disposed within a control room 27, which is separated from the examination room 25. In particular the control room 27 is decoupled from the examination room 25 with respect to propagation of magnetic fields and/or electromagnetic radiation. Usually a physical barrier represented by the dashed lines is present.

The system control computer 26 centrally controls the magnetic resonance scanner 12, such as to execute a predetermined imaging gradient echo sequence. In addition the system control computer 26 includes an evaluation processor (not shown in detail) for evaluation of medical imaging data that is detected during a magnetic resonance examination. Furthermore the medical imaging device 10 includes an operator console 28, which is connected to the system control computer 26 and that is disposed within the control room 27. Control information, such as imaging parameters, and reconstructed magnetic resonance images, can be presented on an output interface 29, for example on at least one display monitor, of the operator console 28 for medical operating personnel 30. Furthermore the operator console 28 has an input interface 31, via which information and/or parameters can be entered during a measurement process by the medical operating personnel 30.

For exchanging data between the medical imaging apparatus 10, namely the magnetic resonance apparatus, and a user, such as one of the medical operating personnel 30, the medical imaging device 10 additionally has a position data evaluation processor 32 and a user interface 34 with a position detector 33 and a display 35. The user interface 34 with the display 35 and the position detector 33 is disposed within the examination room 25.

The position data evaluation processor 32 is included in the system control computer 26 and is disposed within the control room 27. For exchange of data between the position data evaluation processor 32 and the position detector 33, and exchange of data between the position data evaluation processor 32 and the display 35, the medical imaging device 10 has a data transmission bus not shown in any greater detail.

The position detector 33 is designed to detect position data of the patient 22. The position detector 33 includes a 2D camera or a 3D camera. As an alternate embodiment of the invention, for the position detector 33 can include two or more 2D cameras and/or two or more 3D cameras.

The position detector 33, namely the 2D camera or the 3D camera, is disposed within the examination room 25. Here the position detector 33, namely the 2D camera or the 3D camera, is disposed within the examination room 25 such that the patient 22 is in the field of view of the position detector 33 during a preparation in which the patient 22 is already disposed on the patient table 24 of the patient support 23. In the exemplary embodiment, the position detector 33 is disposed on a ceiling area within the examination room 25 such that the detection area 43 of the position detector 33 covers an area in front of the front side of the scanner 12. The position detector 33 is disposed within the examination room 25 that movement and/or the position of the user, especially of the medical operating personnel 30, can be detected during a preparation of the patient 22 for a medical imaging examination, such as a magnetic resonance examination, and/or during the medical imaging examination. For preparation of the patient 22 for the medical imaging examination, and/or during the medical imaging examination, the user and the patient 22 are primarily located in an area that is disposed in the direction of an introduction movement 42 of the patient table 24 into the patient receiving area 20 in front of the front side of the scanner 12. Other arrangements of the position detector 33 that differs therefrom are readily conceivable.

The display 35 includes a touch-screen display 41 that, in the present exemplary embodiment, is disposed on a front side of the housing 21. The touch-screen display 41 can be integrated within a recess of the front side. Preferably the touch-screen display 41 is disposed removably on the housing 21, so that the medical operating personnel 30 can put the touch-screen display 41 down on a working area, for example on the patient table 24 of the patient support 23, so that it can be reached more easily. It is also conceivable for the touch-screen display 41 to be disposed, or to be able to be disposed, on the patient support device 23.

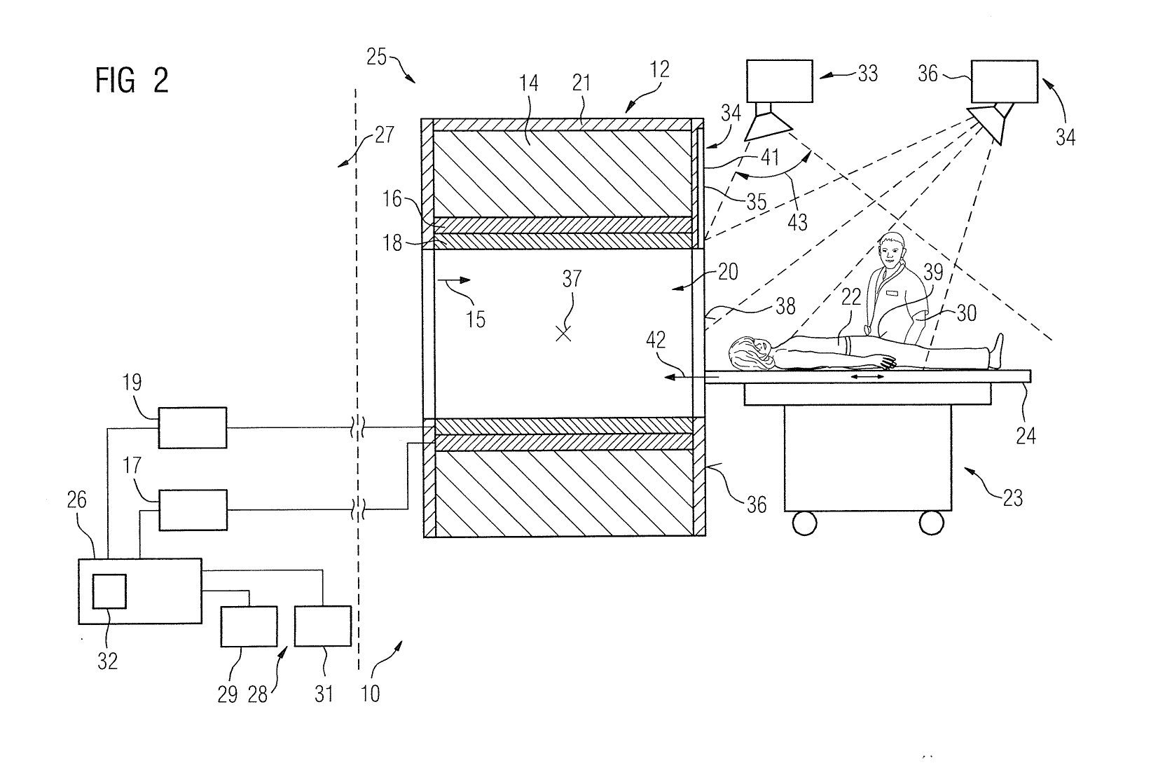

FIG. 2 shows an alternate exemplary embodiment of the medical imaging device 10. Components, features and functions that essentially remain the same are basically labeled with the same reference characters. The description below is essentially restricted to the differences from the exemplary embodiment in FIG. 1, wherein, for components, features and functions that remain the same, the description of the exemplary embodiment in FIG. 1 applies.

The medical imaging device 10 shown in FIG. 2 is likewise formed by a magnetic resonance apparatus. In the embodiment of the medical imaging device 10 shown in FIG. 2 the scanner 12, the system control computer 26, the patient support 24 and the position detector 33 correspond to the embodiment of the medical imaging device 10 described in FIG. 1.

The medical imaging device 10 in FIG. 2 differs from the medical imaging device 10 in FIG. 1 with respect to the design of the user interface 34, particularly the display unit 35 thereof. The display unit 35 in FIG. 2, in addition to the touch-screen display 41, has a projector 36 that is designed for projection of information on one or both of projection surfaces 38 and 39. In the exemplary embodiment, the projector 36 is likewise disposed at the ceiling of the examination room 25. An arrangement of the projector 36 within the examination room differing therefrom is readily conceivable.

The projection surfaces 38 and 39 are a front surface 38 of the housing 21 and/or a patient surface 39. Preferably the projection surfaces 38 and 39 are determined, in accordance with the viewing direction and/or a work sequence of the medical operating personnel 30, by the position data evaluation processor 32 on the basis of the position data detected by the position detector 33.

In an alternate embodiment of the invention, the medical imaging device 10 can have a display unit 35 that has a projector 36, but no additional touch-screen display 41, so that information can be communicated to the medical operating personnel 30 within the examination room 25 exclusively by the projector 36.

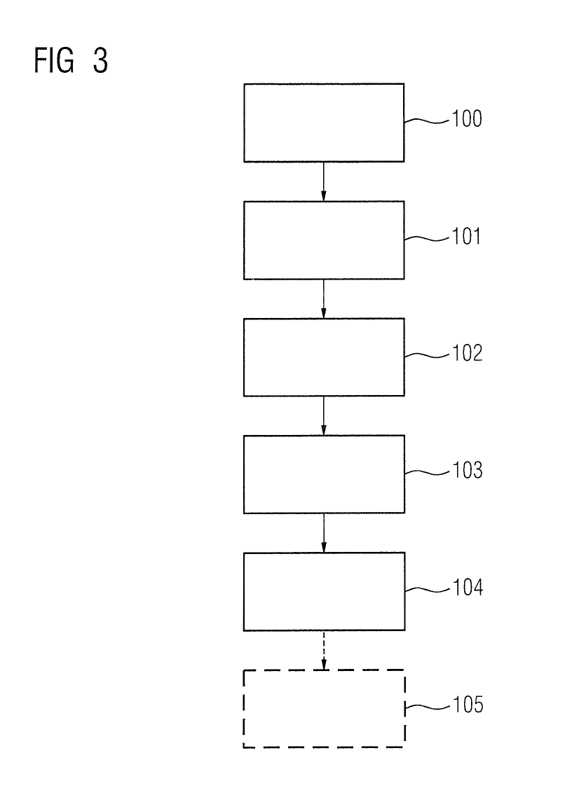

FIG. 3 shows an inventive method for selection of at least one item of examination information for the medical imaging examination the medical imaging examination commences. The method is carried out by the position data evaluation processor 32 together with the user interface 34, especially the position detector 33 and the display unit 35. To this end the position data evaluation processor 32 has corresponding software and/or computer programs, which are stored in a memory of the system control computer 26, especially of the position data evaluation processor 32 (not shown).

In a first method step 100 position data of the patient 22 are detected by the position data detector 33. To this end the patient 22 is already positioned and/or disposed on the patient table 24 of the patient support 23 and the position data represent position information of the patient 22 in relation to the patient table 24.

In a method step 101 subsequent thereto, the detected position data are evaluated by the position data evaluation processor 32. For this purpose, the detected position data are transmitted from the position detector 33 to the position data evaluation processor 32. On the basis of the detected position data, a surface image of the patient 22 is determined by the position data evaluation processor 32. On the surface image, the patient 22 is shown on the patient table 24. Additional units already disposed on and/or at the patient 22 can be displayed on the surface image. On the basis of the detected position data, a location and/or position of the patient 22 can also be determined, which is recognizable in the surface image of the patient 22. The location and/or position of the patient 22 can be, for example, his or her lying on his or her back, in which the patient is disposed with his or her back on the patient table 24, or lying on his or her stomach, in which the patient 22 is disposed with his or her stomach on the patient table 24. In addition the location and/or position of the patient 22 can also be a “head-first” position, in which the patient 22 is moved head first into the patient receiving area 20 for the medical imaging examination, or a “feet-first” position, in which the patient 22 is moved feet first into the patient receiving area 20 for the medical imaging examination.

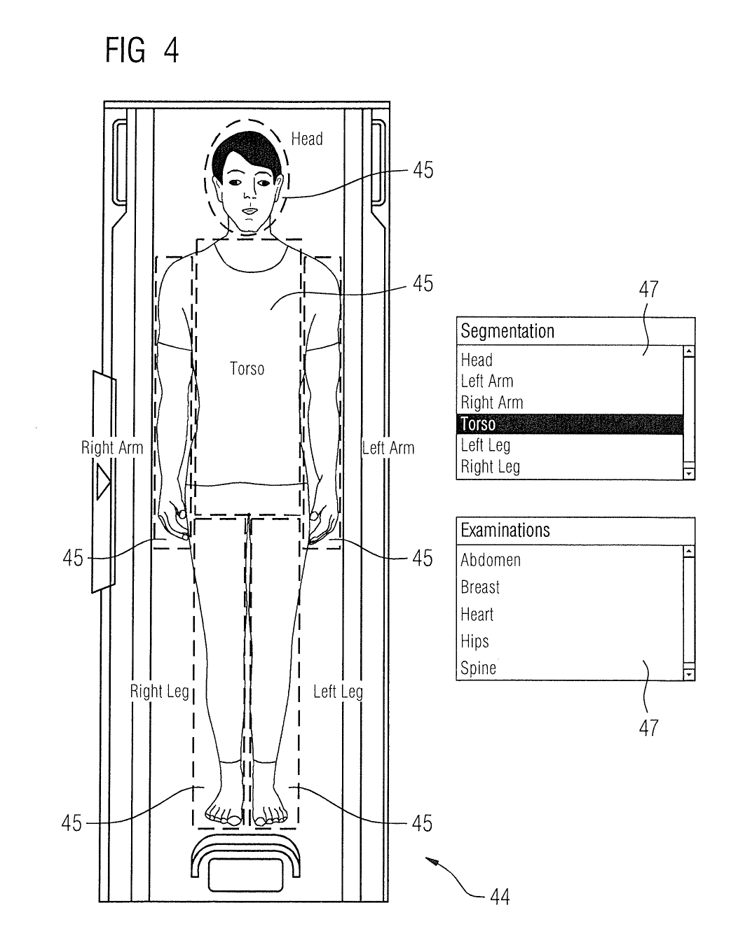

In a further method step 102, a number of items of examination information are determined by the position data evaluation processor 32 on the basis of the surface image. The number of items of examination information can for example include different regions of the body of the patient 22. A segmented surface image 44 of the patient 22 is created, for example, by the position data evaluation processor 32, wherein the segmented surface image 44 is determined on the basis of the surface image of the patient 22 by the position data evaluation processor 32. The surface image is segmented into individual segment areas 45, wherein the individual segment areas roughly are individual areas of the body, such as the right leg or the left leg or the right arm or the left arm or the head or the torso of the patient 22. The individual segment areas 45 are thus roughly adapted to macroscopic anatomy of the patient 22. The number of items of examination information includes the segment areas 45 of the segmented surface image 44. For easier distinction of the individual segment areas 45, these can also be marked differently, for example with different colors. In addition the individual segment areas 45 can also be selected via a text selection 47. The text selection 47 in this case can contain the segment areas 45 and/or also a selection of examination areas, as is shown in FIG. 4. Basically just a single selection option or a combination of just two or three displayed selection options for selection of segment areas 45 is also readily conceivable.

As an alternative or in addition the number of items of examination information can for example also include a maximum area specification 46 for at least one overview measurement, which can be set by the medical operating personnel 30. In addition the number of items of examination information can also include assistance and/or instructions for next workflow steps in the patient preparation. For example the number of items of examination information can include instructions for attaching additional units and/or a possible selection of additional units.

In a further method step 103, the number of items of examination information is displayed by the display 35 for the medical operating personnel 30. The information can be displayed here exclusively by the touch-screen 41 of the display 35, as is shown in FIG. 1. As an alternative or in addition the information can also be displayed via the projector 36 of the display 35, as is shown in FIG. 2. Here the display of the number of items of examination information comprises a projection of the number of items of examination information on one of the projection surfaces 38, 39. In this case the projection surfaced 38 or 39 of the display 36 can be the housing unit 21 of the medical imaging device 10 and/or the patient surface.

The projection of the number of items of examination information on the patient surface can also be undertaken true-to-scale in the further method step 103 for at least one of the number of items of examination information. For example a true-to-scale display of a subdivision of the surface image 44 of the patient 22 into individual segment areas 45 can be projected on the patient 22, cf. FIG. 4. In addition, for definition of an examination area, one or more area settings can be projected true-to-scale in at least one direction onto the patient 22. Preferably an examination area to be set can be projected onto the area of the body of the patient 22 to be examined by the projector 36.

The number of items of examination information is preferably displayed as text information and/or as graphical symbols. For example the individual segment areas 45 within the surface image 44 can be displayed by means of graphical symbols, such as for example by means of areas within the surface image 44 marked in color for the individual segment areas 45. As an alternative or in addition hereto the individual segment areas 45 can also be displayed by means of an output and/or display of text information. For example the individual text information can comprise “right arm” or “left arm” or “right leg” or “left leg” etc. (FIG. 4), from which in a next method step 104 a selection can be made by the medical operating personnel 30.

In the further method step 104 a selection is made of at least one item of examination information for the impending medical imaging examination, wherein the at least one item of examination information comprises a selection from the number of items of examination information. The at least one item of examination information is selected manually by the medical operating personnel 30. In particular the selection of the at least one item of examination information is made by the medical operating personnel 30 by the user interface 34, especially by means of a selection gesture on the touch-screen display 41 and/or by a selection gesture that is detected by the position detector 33. If the number of items of examination information is displayed by means of the projector 36, a detection of a selection gesture for selecting the at least one item of examination information is also undertaken by means of the position detector 33. The position data evaluation processor 32 is designed to detect and/or to recognize a selection gesture of the medical operating personnel 30 and distinguish it from further gestures of the medical operating personnel 30, so that misinterpretations can be avoided. To this end the position data evaluation processor 32 includes the necessary software and/or computer programs, which are stored in the memory unit not shown in any greater detail and are executed on the processor.

The selection the at least one item of selected examination information can in this case comprise a selection of a segment area 45 on the basis of the surface image 44 of the patient 22, where the number of items of examination information available for selection comprises the number of segment areas 45 of the surface image 44, cf. FIG. 4.

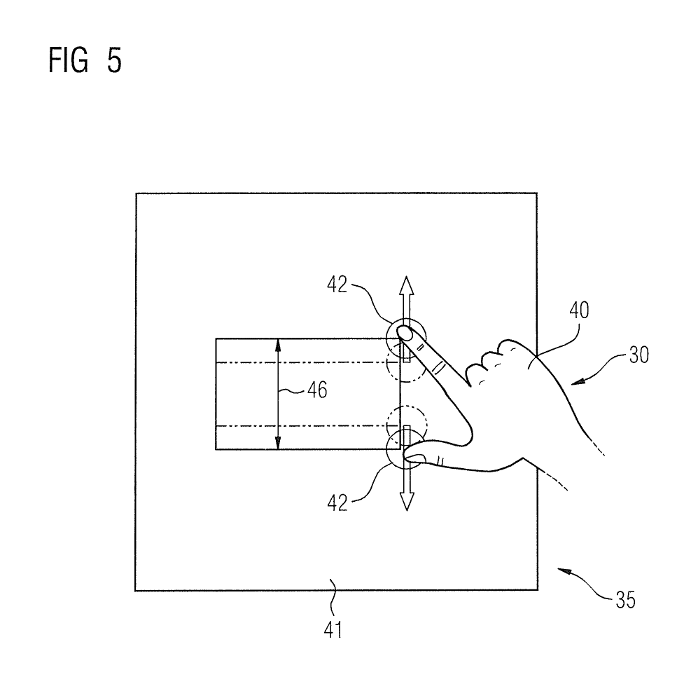

Furthermore the at least one item of selected examination information can also be a selection of an examination region, where the number of items of examination information available for selection comprises a number of examination regions and/or segment areas 45. Here an examination region can be delimited by the medical operating personnel 30 in at least one dimension. This can be done by simple and intuitive hand gestures of the medical operating personnel 30 on an input surface of the touch-screen display 41, cf. FIG. 5. On the touch-screen display 41 a desired examination area can be expanded or pinched with thumb and forefinger of a hand 40 of the medical operating personnel 30 by enlarging or reducing the distance between the two digits. The touch-screen display 41 here includes graphical operating elements 47, which can be displayed on the touch-screen display 41 for the medical operating personnel 30 and which preferably are shown superimposed on the surface image of the patient 22. As an alternative this selection can also be made on one of the projection surfaces 38, 39.

The examination region can be defined here especially for at least one overview measurement and/or at least one localizer measurement. The examination region can be defined here by the medical operating personnel 30 and subsequently a number of overview measurements and/or localizer measurements can be determined independently and/or automatically by the position data evaluation processor 32. If for example the examination area is so large that a single overview measurement and/or localizer measurement is not sufficient, this is recognized automatically by the position data evaluation processor 32 and the selected examination area is divided into two or more overview measurements and/or localizer measurements. Subsequently this division can be shown to the medical operating personnel 30 once more at the display 35 for approval.

Then, on the basis of selected examination information, in the further, optional method step 105, at least one item of information relevant for the medical imaging examination can be generated and displayed for the medical operating personnel 30. The at least one item of information relevant for the medical imaging examination can include assistance for further preparation of the patient 22 here, which predetermines and/or suggests how to proceed further in relation to the selection made. For example in a selection of an examination area and/or a segment area 45, there can be a suggestion for attaching and/or positioning of additional units, such as especially local radio-frequency antenna units, or instructions about how to correctly connect the additional units.

Furthermore the at least one item of information relevant for the medical imaging examination can include at least one examination protocol. On the basis of the selected segment area 45 and/or the selected examination area, a selection of examination protocols can be called up for the medical operating personnel 30 by the position data evaluation processor 32 and displayed at the display 35. This enables a selection of possible examination protocols that can be used for the impending medical imaging examination to be sensibly restricted in relation to the selected segment area 45 and/or the selected examination area. In this way the medical operating personnel 30 can select one of the possible examination protocols in a simple and time-saving manner.

It is also further conceivable, on the basis of the selected segment area 45 and/or the selected examination area, for there to be a definition of an isocenter area for the medical imaging examination. Suggestions that are determined by the position data evaluation processor 32 can also be displayed to the medical operating personnel 30 here by means of the display unit 35. In addition it is also conceivable for the medical operating personnel 30 to select the isocenter area without a suggestion by the position data evaluation processor 32 and to define said area by means of the touch-screen display 41 in the surface image of the patient 22. As an alternative hereto, the isocenter area can also be defined by the projector 36 and the position detector 33.

This method makes it possible to carry out a complete patient preparation within the examination room 25. A start signal of the examination, for example an overview measurement and/or localizer measurement, can already be detected within the examination room 25 by the display 35. The start signal can be entered manually by the medical operating personnel 30 on the touch-screen 41 and/or by the projector 36 and the position detector 33.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for selecting at least one item of examination information or operating a medical imaging apparatus in a diagnostic examination of a patient in order to acquire diagnostic imaging data from the patient, comprising:
in a planning procedure prior to said diagnostic examination, operating a position detector to acquire position data of the patient while the patient is disposed on a patient support of a medical data acquisition scanner for said diagnostic examination;
providing said position data to an evaluation processor and, in said evaluation processor, generating a patient image that comprises a surface image of the patient;
in said evaluation processor, automatically determining multiple items of examination information from said surface image;
presenting said multiple items of examination information to a user at a user interface; and
via said user interface, selecting at least one item of examination information from among said multiple items of examination information presented at said interface, and operating said medical data acquisition scanner in said diagnostic examination according to the selected at least one item of examination information.

2. A method as claimed in claim 1 wherein said user interface is touch-screen display, and presenting said multiple items of examination information as a visual display at said touch-screen display, and receiving said selection of said at least one item of examination information as a touch made at said touch-screen display.

3. A method as claimed in claim 1 wherein said user interface comprises a projector, and presenting said multiple items of examination information as a projection made by said projector onto a projection surface.

4. A method as claimed in claim 3 comprising orienting said projector so that said projection surface is a surface of the patient.

5. A method as claimed in claim 4 comprising projecting said multiple items of examination information on the patient with at least one of said multiple items of examination information being projected true-to-scale on the patient.

6. A method as claimed in claim 3 comprising selecting said at least one item of examination information by detecting a gesture made by said user on or at said projection surface.

7. A method as claimed in claim 1 comprising displaying said multiple items of examination information via said interface represented as at least one of text information and graphical symbols.

8. A method as claimed in claim 1 comprising, in response to said selected at least one item of examination information, presenting an item of information that is relevant for operating said medical data acquisition scanner in said diagnostic examination.

9. A method as claimed in claim 8 comprising presenting said at least one item of information that is relevant for said operation of said medical data acquisition scanner as a prompt for preparing said diagnostic examination.

10. A method as claimed in claim 8 comprising presenting said at least one item of information relevant for operation of said medical data acquisition scanner as an examination protocol.

11. A method as claimed in claim 8 comprising presenting said at least one item of information relevant for operation of said medical data acquisition scanner as an instruction for positioning a component on the patient to be used in said diagnostic examination.

12. A method as claimed in claim 8 comprising presenting said at least one item of information relevant for operation of said medical data acquisition scanner as a designation of an examination region of the patient for said diagnostic examination.

13. A method as claimed in claim 12 comprising designating said examination region of the patient with a limitation of said examination region in at least one dimension.

14. A method as claimed in claim 13 comprising designating said limitation in said at least one dimension of said examination region for an overview scan of the examination region.

15. A method as claimed in claim 1 comprising, in said evaluation processor, segmenting said surface image dependent on said position data to obtain a segmented surface image comprising a plurality of segmented areas, and presenting said segmented areas at said interface as said multiple items of examination information.

16. A method as claimed in claim 15 comprising selecting one of said segmented areas as said selected at least one item of examination information.

17. A method as claimed in claim 15 comprising presenting said segmented areas at said interface with respectively different markings.

18. A method as claimed in claim 1 comprising, in said evaluation processor, identifying an isocenter of said medical data acquisition scanner for said diagnostic examination, from said surface image.

19. A medical imaging apparatus comprising:
a medical data acquisition scanner;
a control computer configured to operate the medical data acquisition scanner in a diagnostic examination to acquire diagnostic image data from a patient;
a position detector that is operable in a planning procedure prior to said diagnostic examination, to acquire position data of the patient while the patient is disposed on a patient support of a medical data acquisition scanner for said diagnostic examination;
an evaluation processor provided with said position data, said evaluation processor being configured to generate a patient image that comprises a surface image of the patient;
said evaluation processor being configured to automatically determine multiple items of examination information from said surface image;
said evaluation processor being configured to present said multiple items of examination information at said user interface; and
said user interface being configured to receive a designation that selects at least one item of examination information from among said multiple items of examination information presented at said user interface, and said control computer being configured to operate said medical data acquisition scanner in said diagnostic examination according to the selected at least one item of examination information.

20. A medical imaging apparatus as claimed in claim 19 wherein said interface comprises a projector configured to project said multiple items of examination information on a projection surface.

21. A medical imaging apparatus as claimed in claim 20 wherein said projector is oriented to project said multiple items of examination information in a surface of the patient as said projection surface.

22. A medical imaging apparatus as claimed in claim 19 wherein said interface comprises a touch-screen display at which said multiple items of examination information are displayed and that receives said selected at least one item of examination information by a touch made at said touch-screen display.

23. A medical imaging apparatus as claimed in claim 19 wherein said position detector comprises a camera, selected from the group consisting of 2D cameras and 3D cameras.

24. A non-transitory, computer-readable data storage medium encoded with programming instructions, said storage medium being loaded into a control computer of a medical imaging apparatus comprising a medical data acquisition scanner and a position detector, said programming instructions causing said control computer to:
operate said medical data acquisition scanner in a diagnostic examination to acquire diagnostic medical data from a patient;
in a planning procedure prior to said diagnostic examination, operate a position detector to acquire position data of the patient while the patient is disposed on a patient support of a medical data acquisition scanner for said diagnostic examination;
generate a patient image that comprises a surface image of the patient;
determine multiple items of examination information from said surface image;
present said multiple items of examination information to a user at a user interface; and
received a selection input via said user interface that selects at least one item of examination information from among said multiple items of examination information presented at said interface, and operate said medical data acquisition scanner in said diagnostic examination according to the selected at least one item of examination information.
